(19) United States
(12) Patent Application Publication
(10) Pub. No.: US 2006/0004346 A1
(43) Pub. Date: Jan. 5, 2006

(54) Bend relief

(76) Inventor: John Donald Begg, Herts (GB)

Correspondence Address: KAPLAN GILMAN GIBSON & DERNIER L.L.P., 900 ROUTE 9 NORTH, WOODBRIDGE, NJ, 07095, US

(21) Appl. No.: 11/154,768

(22) Filed: Jun. 16, 2005

(30) Foreign Application Priority Data
Jun. 17, 2004 (AU) 2004903303

(51) Int. Cl. A61M 25/00

(52) U.S. Cl. 604/525

(57) ABSTRACT

This invention comprises a bend relief for supporting a cannula wherein said bend relief includes a tubular body having a region near a first end that is substantially flexible and a region near a second end that is substantially rigid. Said tubular body has a series of weakened zones disposed therealong, wherein said weakened zones provide said tubular body with regions of differential flexibility.

### FIELD OF INVENTION

The present invention relates to improvements in bend reliefs for supporting cannulae.

### BACKGROUND

Recent advances in medical technology have lead to the creation of many new implantable medical devices. A significant proportion of these implantable medical devices require the use of cannulae.

Prior art cannulae have been constructed of generally soft and flexible material that is generally biocompatible and may encourage cellular seeding. Typically, cannulae have been constructed of velour and/or Dacron™ material assembled into a tube or a conduit. Some cannulae have been configured so that the outer surface of the tube comprises a concertina arrangement. The concertina arrangement allows the body of the cannulae to bend so that the position and attachment points of the cannulae may be varied.

One disadvantage associated with this design of cannulae is that cannulae constructed of soft materials, such velour and/or Dacron™, are prone to tearing or rupturing. Additionally, kinking may occur when the body of a cannula is twisted or subjected to torsional forces and this kinking may also lead to blood flow occlusion within the conduit. Also, the soft flexible conduits of these types of cannulae often experience collapse when a negative pressure is applied to the interior of the graft, which routinely occurs when the conduit is connected to a blood pump.

In the past, there have been many attempts at solving or addressing these problems. One of the most common solutions was to reinforce the cannulae or graft with a bend relief. These bend reliefs are generally required to be flexible to allow positioning and rigid enough to support the interior graft material to prevent collapse, rupture.

U.S. Pat. No. 6,001,056 (Jassawalla et al), discloses a bend relief which supports the structure of the soft and flexible cannula; and also allows the cannula to be flexible enough to be bent. However, one disadvantage of this device is that the bend relief may be too flexible in some situations and this may result in occlusion of the blood flow within the cannula if the bend relief is overly bent. It would be therefore preferable to make some regions of the bend relief stiffer to reduce the possibility of over bending of the bend relief and thereby reducing the risk of blood flow occlusion.

The present invention aims to provide a bend relief for a cannula which overcomes at least one of the disadvantages of the prior art.

### SUMMARY OF INVENTION

According to a first aspect the present invention consists in a bend relief for supporting a cannula, said bend relief including a tubular body having a region near a first end that is substantially flexible and a region near a second end that is substantially rigid, said tubular body having a series of weakened zones disposed therealong, and wherein said weakened zones provide said tubular body with regions of differential flexibility.

Preferably said series of weakened zones includes a plurality of apertures.

Preferably in one embodiment said apertures are slots. Preferably at least one of said slots is disposed at least partially circumferentially about said tubular body.

Preferably the size of said apertures is proportional to the flexibility of said tubular body.

Preferably the density of apertures is proportional to the flexibility of said tubular body.

Preferably said tubular body has a greater density of apertures near said first end than near said second end.

Preferably said apertures disposed near said first end have a greater size than apertures disposed near said second end.

Preferably there is a greater number of apertures near said first end than there is near said second end.

Preferably said cannula is constructed of Dacron™ or velour.

Preferably said second end of said bend relief is adapted to attach to a blood pump.

Preferably said tubular body includes a positioning strip along the longitudinal axis of the tubular body.

Preferably said tubular body is constructed of polypropylene.

### BRIEF DESCRIPTION OF THE FIGURES

Embodiments of the present invention will now be described with reference to accompanying drawings wherein:

FIG. 1 shows cross sectional side view of a first embodiment of the present invention; and

FIG. 2 shows an external side view of the embodiment shown in FIG. 1.

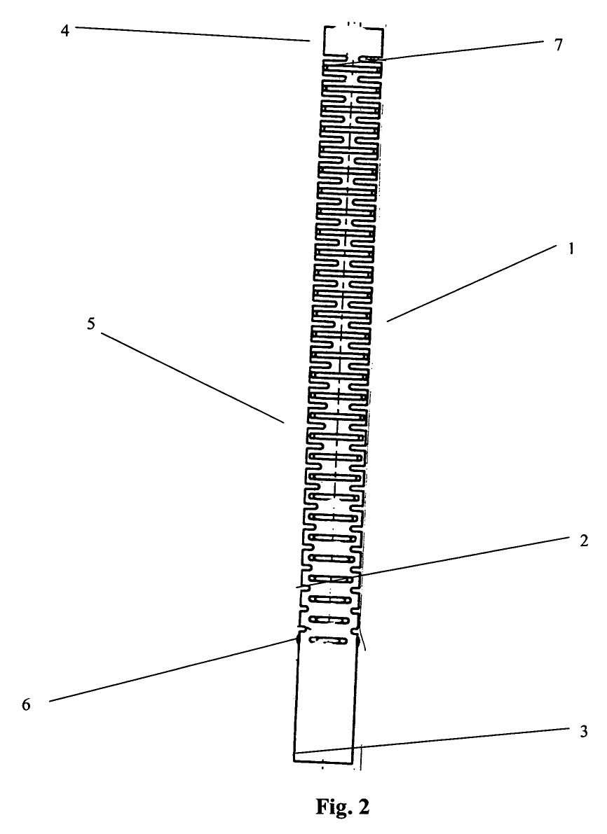

### BRIEF DESCRIPTION OF EMBODIMENTS

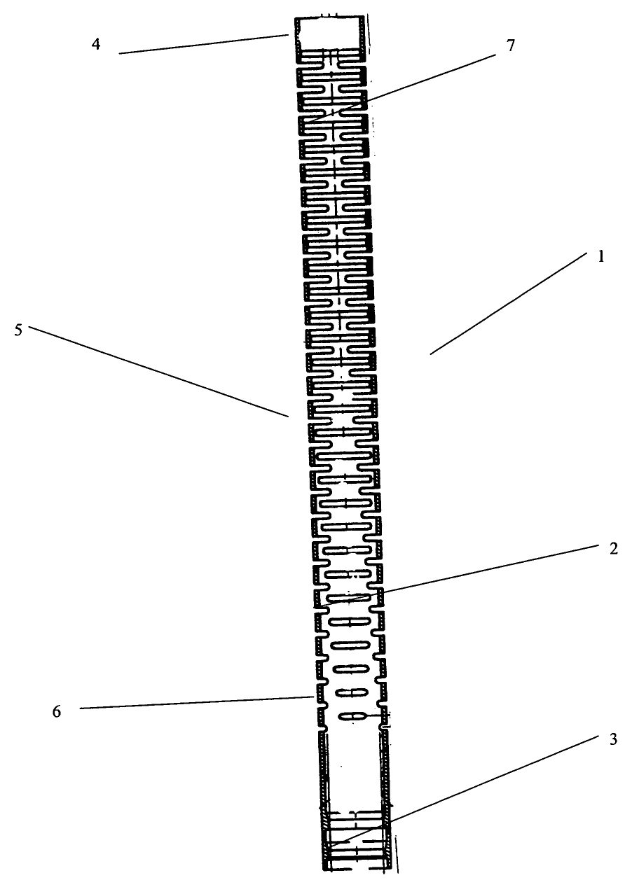

A first embodiment of the present invention is shown in FIGS. 1 & 2. A bend relief 1 is shown and includes: a tubular body 2; a first end 3; and a second end 4. The tubular body 2 is relatively rigid and adapted to receive a graft or cannula (not shown) through the center of the bend relief 1. The tubular body 2 functions to a support the graft or cannula and prevent: kinking, tearing and collapse. The graft or cannula is preferably constructed of a soft and flexible material, such as velour or Dacron™, and may include a concertina fold arrangement to allow for uniform bending of the cannula.

Preferably, this embodiment may be for use with an implantable medical device, such as the cannulae that work with implantable blood pumps. The first end 3 of the bend relief 1 may be adapted or modified to engage with the outlet of an implantable blood pump (not shown). Whilst bend relief 1 is for use with the cannulation of a blood pump, it is preferable that bend relief 1 be relatively rigid proximal to the blood pump and be more flexible at a position distal from the blood pump, such as in the region of the second end 4. This configuration may prevent kinking of the cannulae proximal to the blood pump and thereby prevent accidental occlusion of the blood flow.

Additionally, the more flexible bendable region at the second end 4 allows the cannula to be manipulated to suit the physiological needs and requirements of the patient. For example, the bend relief 1 may used to cover a cannula from the outlet of a blood pump to an artery such as the aorta. The artery may be in a slightly different anatomical position depending on the patient, or the attachment location of the cannula may differ depending on the patient's needs. The flexibility in the region of the second end 4 of the bend relief 1 will allow the bend relief 1 and the cannula to be adapted to the implantation circumstances.

The bend relief 1 preferably includes a series of slots 5. These slots 5 may be partially circumferential relative to the tubular body 2. Slots 5 weaken the rigidity of the tubular body 2 in localised regions and increase the flexibility in these regions. In the preferred embodiment shown in FIG. 1, the bend relief 1 includes a series of horizontal slots 5. Each series of slots 5 extends down each side, top and bottom of the tubular body 2. In this embodiment, the slots 5 are arranged so as to allow the bend relief 1 to bend in all directions.

This embodiment also shows regions of differential flexibility. The region proximal to the first end 3 in this embodiment is relatively less flexible when compared to the second end 4. This is accomplished by the use of the series of slots 5 to differentially weaken the rigidity of the tubular body 2. The tubular body 2 is increasingly weakened where the slots 7 are of a relatively larger size and less weakened where there are smaller sized slots 6. Therefore, the regions of the tubular body 2, which include relatively larger sized slots 7, have increased flexibility and bendability. In FIG. 1, the smaller sized slots 6 are proximal to the first end 3 and the larger sized slots 7 are proximal to the second end 4. Additionally, there is a relatively smooth transition between the smaller sized slots 6 and the larger sized slots 7. This is preferred so as to allow an even transition of flexibility to further prevent kinking.

In further embodiments of the present invention, the series of slots 5 along the tubular body 2 of the bend relief 1 may be replaced with other features which may differentially weaken the bend relief 1 to increase the flexibility in desired regions. These other features may include modified slots or holes, which have a similar functional effect to the series of slots 5 in FIG. 1.

Additionally, the number of holes or slots may be modified to facilitate to increase or decrease flexibility of the tubular body 2 in selected regions. Another modification may also achieve a similar result by varying the density of holes or slots in the tubular body 2.

Preferably, the bend relief 1 may be constructed of polypropylene, although any relatively rigid biocompatible material that can be selectively weakened may be used. The bend relief 1 preferably has constant diameter of approximately 1.5 cm and an approximate length of 18 cm.

The above descriptions describe only some of the embodiments of the present invention.

Modifications may be obvious to those skilled in the art and may be made without departing from the scope and spirit of the present invention.

The term “comprising” (and its grammatical variations) as used herein is used in the inclusive sense of “having” or “including” and not in the exclusive sense of “consisting only of”.

1. A bend relief for supporting a cannula, said bend relief including a tubular body having a region near a first end that is substantially flexible and a region near a second end that is substantially rigid, said tubular body having a series of weakened zones disposed therealong, and wherein said weakened zones provide said tubular body with regions of differential flexibility.

2. The bend relief as claimed in claim 1, wherein said series of weakened zones includes a plurality of apertures.

3. The bend relief as claimed in claim 2, wherein said apertures are slots.

4. The bend relief as claimed in claim 3, wherein at least one of said slots is disposed at least partially circumferentially about said tubular body.

5. The bend relief as claimed in claim 2, wherein the size of said apertures is proportional to the flexibility of said tubular body.

6. The bend relief as claimed in claim 2, wherein the density of apertures is proportional to the flexibility of said tubular body.

7. The bend relief as claimed in claim 1, wherein said tubular body has a greater density of apertures near said first end than near said second end.

8. The bend relief as claimed in claim 1, wherein said apertures disposed near said first end have a greater size than apertures disposed near said second end.

9. The bend relief as claimed in claim 1, wherein there is a greater number of apertures near said first end than there is near said second end.

10. The bend relief as claimed in claim 1, wherein said cannula is constructed of Dacron™ or velour.

11. The bend relief as claimed in claim 1, wherein said second end of said bend relief is adapted to attach to a blood pump.

12. The bend relief as claimed in claim 1, wherein said tubular body includes a positioning strip along the longitudinal axis of the tubular body.

13. The bend relief as claimed in claim 1, wherein said tubular body is constructed of polypropylene.
